SEQUENCE LISTING (3 sequences)

SEQ ID NO: 1 - protein, 8 aa, Artificial Sequence
  FLAG
DYKDDDDK

SEQ ID NO: 2 - protein, 10 aa, Artificial Sequence
  Myc
EQKLISEEDL

SEQ ID NO: 3 - protein, 9 aa, Artificial Sequence
  HA
YPYDVPDYA

(19) United States
(12) Patent Application Publication
(10) Pub. No.: US 2012/0021998 A1
(43) Pub. Date: Jan. 26, 2012

(54) Cancer Diagnosis and Treatment Methods of Use

(75) Inventors: Show-Li Chen, Taipei (TW); Hsuan-Hao Chen, Taipei (TW); Kuan-Liang Lin, Taipei (TW); Hsin-Hsiung Chen, Taipei (TW); Szu-Wei Chang, Taipei (TW)

(73) Assignee: NATIONAL TAIWAN UNIVERSITY, Taipei City (TW)

(21) Appl. No.: 12/839,754

(22) Filed: Jul. 20, 2010

(51) Int. Cl. A61K 38/16; G01N 33/574; A61P 35/00

(52) U.S. Cl. 514/19.5; 435/7.23

(57) ABSTRACT

The present invention relates to a method for treating a subject suffering from growth of AR-containing tumor cells, comprising administrating the subject an effective amount of DDB2. The present invention also relates to a kit for the diagnosis of cancer.

### FIELD OF THE INVENTION

The present invention relates to a tumor-suppressing method. The present invention also relates to a kit for the diagnosis of cancer. In summary, the present invention is about the use of damaged-DNA binding protein 2 (DDB2) in tumor diagnosis and treatment.

### BACKGROUND OF THE INVENTION

Recently, the male prostate cancer incidence increased year after year. According to the report of the Department of Health (DOH) of Taiwan, prostate cancer ranked seventh in both top ten fatal cancers and male top ten fatal cancers and cannot be ignored since it's minacity to males. Prostate cancer is formed due to malign hypertrophy of prostate cells, which might relate to genetic factors, lack of male hormones, diet, environmental factors and viral or bacterial infections.

The early symptom of prostate cancer is similar to which of benign prostatic hypertrophy (BPH). In early phase, patients feel difficultly to urinate or micturate frequently. When the urinary tract or the bladder neck gets invaded or obstructed, the symptoms like hematuria, incontinence, intermittent or weak urine flow, pain and burning sensation during urinating or ejaculation and urinary tract infection may appear. Also, prostate cancer often transfers to the bone and then causes bone pain, fractures or symptoms of spinal nerve compression. Prostate cancer can be divided into four phases in accordance with the degree of the distribution cancer cells:

Phase I: Tumor is located in the prostate, and the clinical examination is difficult to find cancer cells.
Phase II: Clinical examination detects prostate cancer in the lump, but the cancer is confined within the prostate capsule.
Phase III: Violation of prostate cancer cells outside the organization, such as: seminal vesicle, peripheral fat, urethra muscles and bladder neck.
Phase IV: Cancer metastasis, violations of the pelvis, lymph nodes or distant organs.

Diagnosis of prostate cancer usually means a “rectal examination”, that is by rectal examination by a physician to feel whether the prostate lumps or nodules phenomenon. Recently, because of advances in diagnostic techniques, the test of blood prostate specific antigen (PSA) and prostatic acid phosphatase (PAP) is widely used in routine medical examinations. This greatly enhances the efficiency of early diagnosis of prostate cancer and thus increases the cure rate. Inevitably, however, there was still a part of the patients who had prostate cancer and were detected late or get recurrence or metastasis after surgical resection.

The clinical treatments of prostate cancer are as follows:
1. Radical surgical treatment: For patients with earlier phase, resection of the prostate gland can eradicate all cancer cells within the prostate; but it is useless for those whose cancer cells metastasize and violate the lymph nodes. The operative mortality is low, but most patients lost their sexuality and suffered from urinary incontinence after the surgical operation.
2. Cryotherapy: Cryotherapy kills cancer cells by freezing them. It is a less aggressive therapy and patients need not to have general anesthesia. This therapy has the better effect than Radical surgical treatment when used in the treatment of cancer recurrence after radical treatment and also reduces postoperative urinary problems; however, it leads to increase the likelihood of sexual dysfunction to 90%.
3. Radiation therapy: This method uses radiation to destroy cancer cells and inhibit their growth and division by the treatment course of about 30 to 40 times of exposure and was usually applied to patients who can't perform the surgery. Radiation therapy is a local treatment that can only kill cancer cells in the treatment region, and the course of treatment will cause side effects and discomfort. There is new radiation treatment method which uses computer-knife radiosurgery for the treatment of localized tumors, and high doses of radiation can be accurately applied to the tumor to minimize side effects and reduce the damage to normal organ function. Course of treatment is without real-knife surgery, and the effect of surgical treatment can be achieved in about 3 to 5 times of operations. However, it is only for the treatment of local and costs much time for a single treatment. Moreover, it costs so much money.
4. Hormone therapy: Hormone therapy is applied to patients who are not suitable for surgical operation because of the metastasis of tumor. Most prostate tumor cells are very sensitive to androgen, and blockade of androgen-tumor cell interaction leads to the inhibition of tumor proliferation and metastasis. Clinically, the most common way of hormone therapy is testes removal or luteinizing hormone-releasing hormone analogues (LHRH analogues) treatment. However, hormone therapy is apt to cause flushing, decreased sexual desire, sexual dysfunction, osteoporosis and other side effects. Also, this therapy is only effective in treatment of one type of prostate cancer (androgen-dependent prostate cancer, ADPC) but not another (androgen-independent prostate cancer, AIPC).

Damaged-DNA binding protein 2 (DDB2, also named p48), a member of DDB 1 and Cu14-associated factors (DCAFs), contains three WD40 domain and was originally found to involve in nucleotide excision repair along with damaged-DNA binding protein 1 (DDB1) (Takao, M., M. Abramic, et al., A 127 kDa component of a UV-damaged DNA-binding complex, which is defective in some xeroderma pigmentosum group E patients, is homologous to a slime mold protein. Nucleic Acids Res 21(17): 4111-8 (1993)). DDB2-deficient mice not only were hypersensitive to UV-induced skin carcinogenesis but also developed a high rate of malignant tumor in internal organ which indicate DDB2 function as a tumor suppressor (Itoh, T., S. Iwashita, et al., Ddb2 is a haploinsufficient tumor suppressor and controls spontaneous germ cell apoptosis. Hum Mol Genet. 16(13): 1578-86 (2007)). In addition to DNA repair, DDB2 may function as a transcription factor to regulate gene expression. It had reported that DDB2 acts as a co-factor of E2F1 (Hayes, S., P. Shiyanov, et al., DDB, a putative DNA repair protein, can function as a transcriptional partner of E2F1. Mol Cell Biol 18(1):240-9 (1998)) and that associated with chromatin-acetylating transcription co-activator STAGA complex (SPT3-TAFII31-GCN5L acetylase complex). In contrast that DDB2 is considered as a tumor suppressor, recent study has reported that DDB2 is a candidate for oncogene in breast cancer which may contribute to breast tumor progression (Kattan, Z., S. Marchal, et al., Damaged DNA binding protein 2 play a role in breast cancer growth.” PLoS One 3(4): e2002 (2008)). However, the relationship between DDB2 and prostate cancer was not disclosed.

Ubiquitin E3 ligase contains two important functions; one for catalysis of isopeptide bond formation and the other for the recruitment of substrates to this catalytic activity. The cullin (CUL) family is evolutionarily conserved proteins that assemble a large family of cullin-dependent E3 ligase. The human cullin family includes CUL1, CUL2, CUL3, CUL4A, CUL4B, CUL5 and CULT. All cullins contain a conserved carboxy-terminal domain which binds to small RING finger protein: ROC1 (Regulator of Cullins-1, also called Rbx 1) or ROC2 (Regulator of Cullins-2) (Petroski, M. D. and Deshaies, R. J., Function and regulation of cullin-RING ubiquitin ligases. Nat Rev Mol Cell Biol 6 (1), 9 (2005)). The small RING finger protein can recruit E2 ubiquitin-conjugating enzyme to proceed to ubiquitination. Cullin-dependent E3 ligase require to interact with an adaptor protein to target specific substrate, rather than binding to substrate directly as other E3 ligase. For example, CUL 1-dependent ligase rely on interaction with an adaptor protein SKP1 (S-phase kinase-associated protein 1) to bridge an F-box protein to target specific substrate (Petroski, M. D. and Deshaies, R. J., Function and regulation of cullin-RING ubiquitin ligases. Nat Rev Mol Cell Biol 6 (1), 9 (2005)). CUL4 fuctions as ubquitin E3 ligase by recruiting ring finger protein (ROC1) and various substrate receptors. To target specific substrate, CUL4 utilizes the C-terminus to bind with ROC1 and the N-terminus to interact with linker protein (DDB1) which recruits various substrate receptors to target specific substrate. A well-known model is DDB2-DDB1-CUL4 complex which involves in NER (nucleotide excision repair) pathway after UV-irradiation. The DDB2-DDB1-CUL4 E3 ligase complex is recruited to the DNA lesion foci at the damaged DNA, and then ubiquitinates histone H2A, and H3, H4 (Wang, H. et al., Histone H3 and H4 ubiquitylation by the CUL4-DDB-ROC1 ubiquitin ligase facilitates cellular response to DNA damage. Mol Cell 22 (3), 383 (2006)) etc. After ubiquitination, the histones may dissolve from the damaged nucleosome that makes the damaged DNA exposed. Later, the NER pathway factor XPC (Xeroderma pigmentosum group C-complementing protein) is recruited to the damaged site and the NER pathway proceeds (Sugasawa, K. et al., UV-induced ubiquitylation of XPC protein mediated by UV-DDB-ubiquitin ligase complex. Cell 121 (3), 387 (2005)).

The turnover of androgen receptor (AR) plays an important role in AR protein regulation. There are three pathways reportedly to be involved in AR degradation. Firstly AR can be phosphrylated by PI3K/AKT and subsequently undergoes ubiquitination by MDM2 E3 ligase. After ubiquitination, AR was degraded through 26S proteasome (Gaughan, L. et al., Tip60 and histone deacetylase 1 regulate androgen receptor activity through changes to the acetylation status of the receptor. J Biol Chem 277 (29), 25904 (2002)). Secondly, androgen-induced AR translocation can be interfered by phosphatidylinositol-3, 4, 5-trisphosphate 3-phosphatase (PTEN). The interaction between AR and PTEN may expose the active site of the AR for the recognition of caspase-3, leading to AR degradation (Lin, H. K., Y. C. Hu, et al., Regulation of androgen receptor signaling by PTEN (phosphatase and tensin homolog deleted on chromosome 10) tumor suppressor through distinct mechanisms in prostate cancer cells. Mol Endocrinol 18(10): 2409-23. (2004)). Thirdly, in DDB1-CUL4B complex, AhR (dioxin receptor) can be activated in the presence of ligand (3-methylcholanthrene) and then interacts with aryl hydrocarbon receptor nuclear translocator (Arnt) to form heterodimer and translocate into nucleus. The heterodimer can associate with DDB1-CUL4B complex to assemble a functional E3 ligase. Sex steroid hormone receptor AR or ER can be a target substrate and ubiqutinated by this this E3 ligase complex (Ohtake, F. et al., Dioxin receptor is a ligand-dependent E3 ubiquitin ligase. Nature 446 (7135), 562 (2007)). However, the relationship between DDB2 and AR was not disclosed.

### SUMMARY OF THE INVENTION

The present invention provides a method for treating a subject suffering from growth of androgen receptor (AR)-containing tumor cells, comprising administrating the subject an effective amount of damaged-DNA binding protein 2 (DDB2).

The present invention also provides a cancer-diagnosing kit, comprising (a) DDB2 and (b) DDB2-biomarker complex.

### BRIEF DESCRIPTION OF THE DRAWINGS

FIG. 1. DDB2 interacts with AR reciprocally in vitro and in vivo. FIGS. 1A and 1B show that DDB2 can directly interact with AR extracellularly. FIG. 1C shows that DDB2 can also interact with AR in vivo.

FIG. 2. DDB2 induces the ubiquitin level of AR and DDB1-CUL4A is involved in AR protein stability. FIG. 2A shows that DDB2 can ubiquitinize AR. FIG. 2B shows that the increase of DDB1 lowers the expression of intracellular AR. FIG. 2C shows that after leveling down the expression of DDB1 by shDDB1, the AR level measures higher than control. FIG. 2D shows that after leveling down the expression of CUL4A by shCUL4A, the AR level measures higher than control. The quantitative data of AR protein was analyzed by UVP and presented as mean+SD.

FIG. 3. Biological significance of DDB2 effects. LNCap and PC-3 cell were plated in 6-well dishes in complete medium, and cell numbers were counted per two days. Surviving cell numbers were counted by trypan blue staining. Means are shown for three experiments. Nuclear and cytoplasmic fractions were prepared from LNCap and PC-3 cells then analyzed by western blot.

### DETAILED DESCRIPTION OF THE INVENTION

The term “DDB2” used herein refers to damaged-DNA binding protein 2, “AR” refers to androgen receptor, “DDB1” refers to damaged-DNA binding protein 1, “CUL4A” refers to cullin 4A protein, “LNCaP” refers to androgen-dependent prostate tumor cell line which contains AR, and “PC-3” refers to androgen-independent prostate tumor cell line which is without AR. The term “GST” used herein refers to glutathione S-transferase, “FLAG” refers to FLAG® epitope “DYKDDDDK” (SEQ ID NO.1), “Myc” refers to c-Myc epitope tag “EQKLISEEDL” (SEQ ID NO.2), and “HA” refers to hemagglutinin epitope tag “YPYDVPDYA” (SEQ ID NO.3). The term “siRNA” used herein refers to small interfering RNA, “shRNA” refers to small hairpin RNA, and “MG132” refers to proteasome inhibitor.

The present invention provides a method for treating a subject suffering from growth of androgen receptor (AR)-containing tumor cells, comprising administrating the subject an effective amount of damaged-DNA binding protein 2 (DDB2).

The protein DDB2 mentioned herein is one subunit of damaged-DNA binding protein complex. It can associate with DDB1 and CUL4A and forming DDB2-DDB1-CUL4A complex.

In the present invention, the DDB2 can interact with androgen receptor. By the participation of DDB1-CUL4A complex, the DDB2-DDB1-CUL4A protein complex ubiquatinates AR and let it degrade, and therefore, the growth of AR-dependent tumor cell is suppressed.

In the present invention, the preferable tumor cell whose growth is suppressed by an effective amount of DDB2 is prostate tumor cell. Prostate tumor cells are divided into two groups according to the presence of AR. In a preferred embodiment of the invention, the prostate tumor cell lines are LNCaP and PC-3; the former is the androgen-dependent prostate tumor cell line while the latter is androgen-independent. In summary, the method of the invention can suppress the growth of LNCaP androgen-dependent (AR-containing) prostate tumor cell line effectively.

In the present invention, the administration can be applied by any known methods. For example: it can be manufactured into liquid injection form and applied to organisms. In addition, the effective amount of DDB2 can also be delivered via oral route. Through the digestive system and circulatory system, it will be delivered to target locations.

The present invention also provides a cancer-diagnosing kit, comprising (a) DDB2 and (b) DDB2-biomarker complex. In the cancer-diagnosing kit of the present invention, the biomarker complex is chosen from the group including radioactive isotopes, fluorescent molecules, luminescent markers, enzymes and affinity molecules. The cancer-diagnosing kit can also contain a signal-detecting reagent chosen from the group including antibodies, enzymes, affinity molecules and chemical coloring elements. In the present invention, the preferable cancer-diagnosing kit is prostate cancer-diagnosing kit.

### EXAMPLES

The examples below are non-limiting and are merely representative of various aspects and features of the present invention.

### Example 1

### Protein-protein Interaction between DDB2 and AR In Vitro and In Vivo

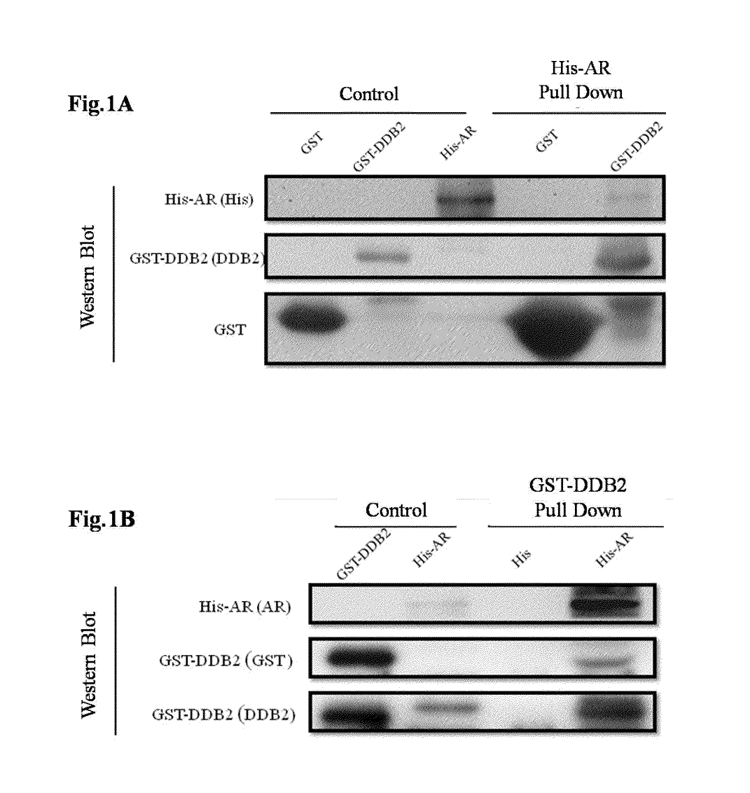

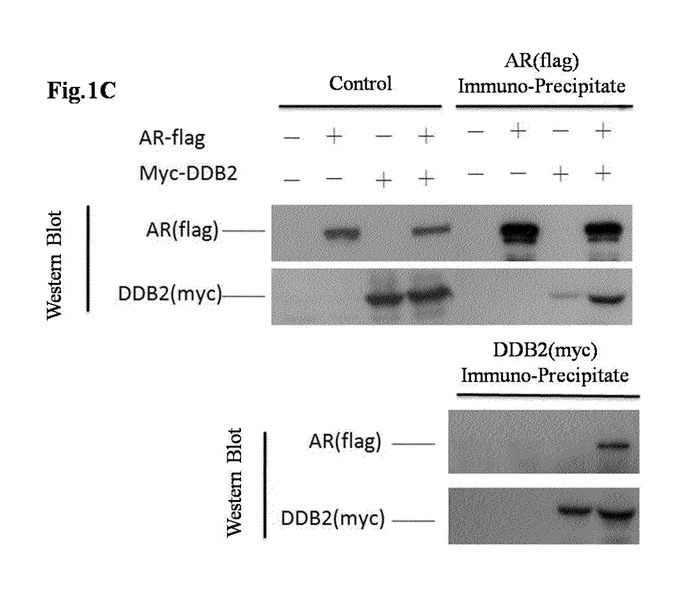

To test whether DDB2 could directly interact with AR reciprocally, His-AR fusion protein synthesized by E. coli was purified using Ni-NTA beads and incubated with purified GST-DDB2 fusion protein in GST pull down assay. The co-purified complex was separated with SDS-PAGE and western-blotting was performed with anti-His for AR or anti-GST for DDB2, respectively (FIG. 1A). Also, the recombinant GST-DDB2 protein was purified and then incubated with purified His-AR protein. The eluted proteins were analyzed by western-blotting (FIG. 1B). The results showed that the AR-bound beads could be pulled down with the purified His-AR protein (FIG. 1A); and vice versa (FIG. 1B). These data indicated that AR directly interacts with DDB2 in vitro.

Furthermore, to test whether DDB2 associated with AR reciprocally in vivo, the Myc-tagged DDB2 and FLAG-tagged AR plasmids were co-transfected into NEK 293T cells by the standard calcium phosphate method. After 48 h, the cell lysate was collected and immuoprecipitated with anti-FLAG to AR or anti-Myc to DDB2, respectively. And then, the co-immunoprecipitated complexes were analyzed with the antibodies indicated. The Western blotting analysis data showed that there was an interaction signal compared with the control group. The same result was also observed by reciprocal experiment (FIG. 1C). These results indicated that DDB2 can interact with AR in vivo. Taken together, DDB2 can interact reciprocally with AR in vitro and in vivo, implying that DDB2 is a novel AR-binding protein.

### Example 2

### Effect of DDB2 on AR Protein Ubiquitination

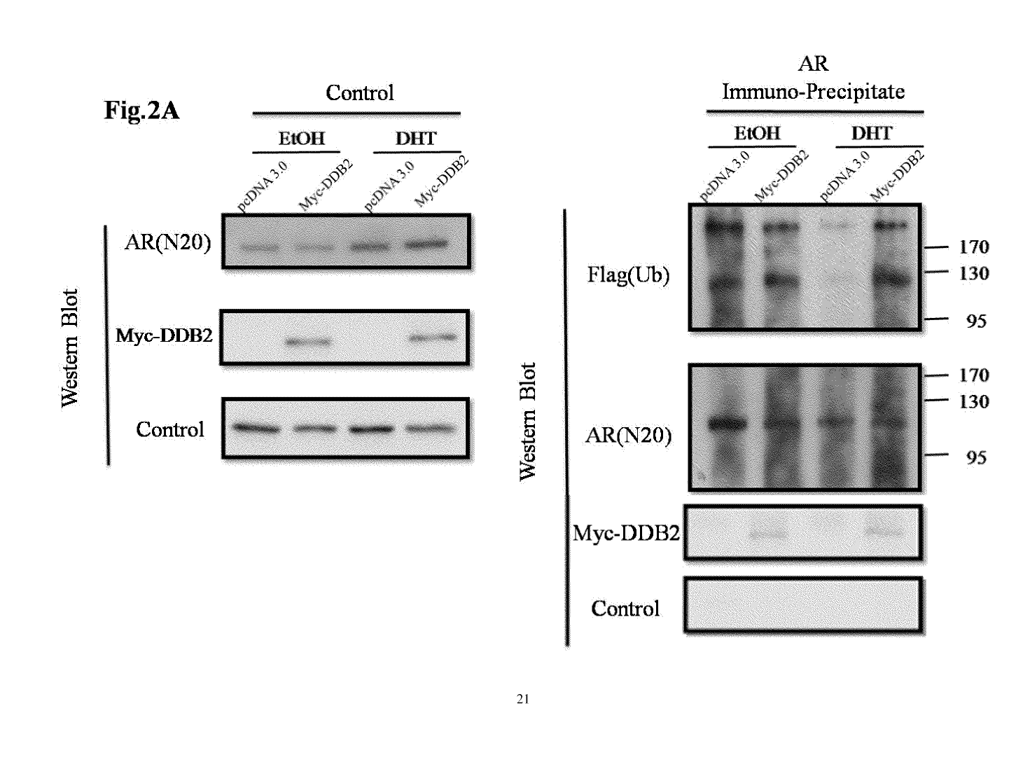

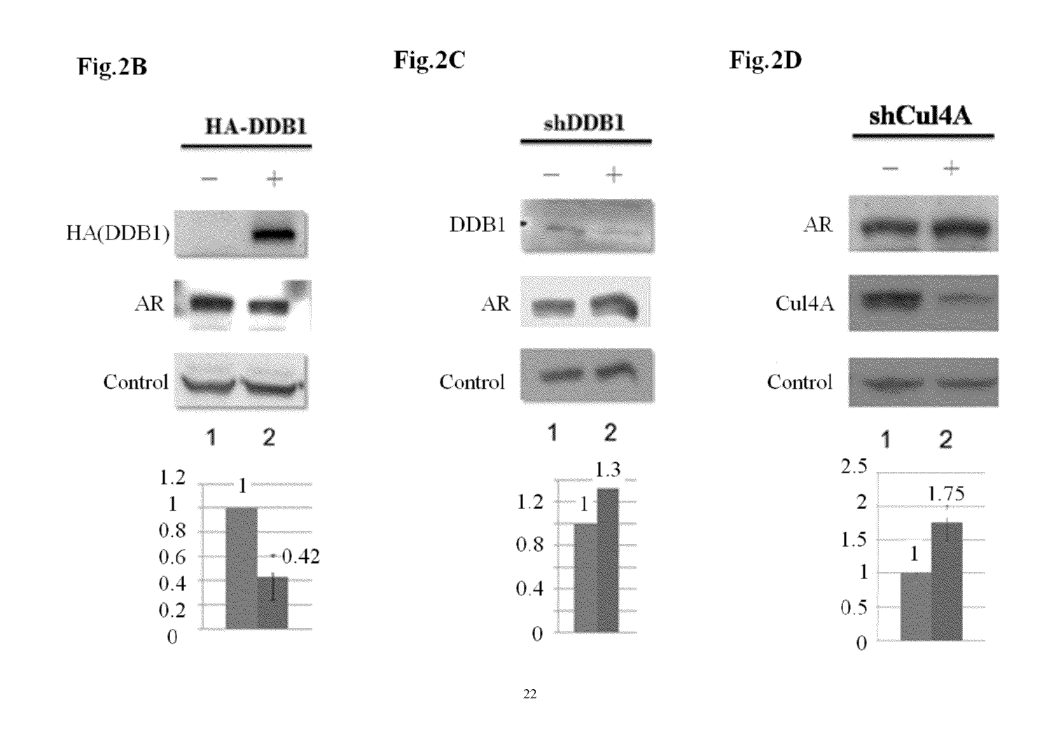

To further investigate the effect of DDB2 on ubiquitylation level of AR, equilmolar amounts of AR (10 μg), Myc-tagged DDB2 (10 μg) and FLAG-tagged ubiquitin (5 μg) was transfected into NEK 293T cells. After 48 hours, the cell were treated with 20 μM MG132 for 6 hours incubation. The cell lysate was used to proceed with immunoprecipitation with anti-AR antibody and then subjected to Western blotting analysis with anti-FLAG antibody to examine the ubiquitylation level of AR protein. The result showed that DDB2 can induce AR ubiquitin level compared with vector control (FIG. 2A).

Recently, it has been reported that DDB1-Cu14 E3 ligase was involved in the degradation of AR protein and DDB2 was found to be a member of DCAFs which can interact with DDB1 and functions as a substrate receptor of DDB1-Cul4 E3 ligase complex. In this regard, DDB1 was expressed in prostate cancer cell line, LNCaP to test whether DDB2-degrading AR involves in DDB1-CUL4 degradation pathway. The result showed that DDB1 can reduce AR protein level (FIG. 2B) Moreover, siRNA was used to knockdown endogenous DDB1 (FIG. 2C) or Cu14A (FIG. 2D), and AR was increased in siDDB1 or siCUL4A expressing cells in response. These data suggest that DDB1-Cul4A pathway might be involved in AR degradation.

### Example 3

### Overexpression of DDB2 Results in Reducing Growth Rate of Androgen-dependent Prostate Cancer Cells

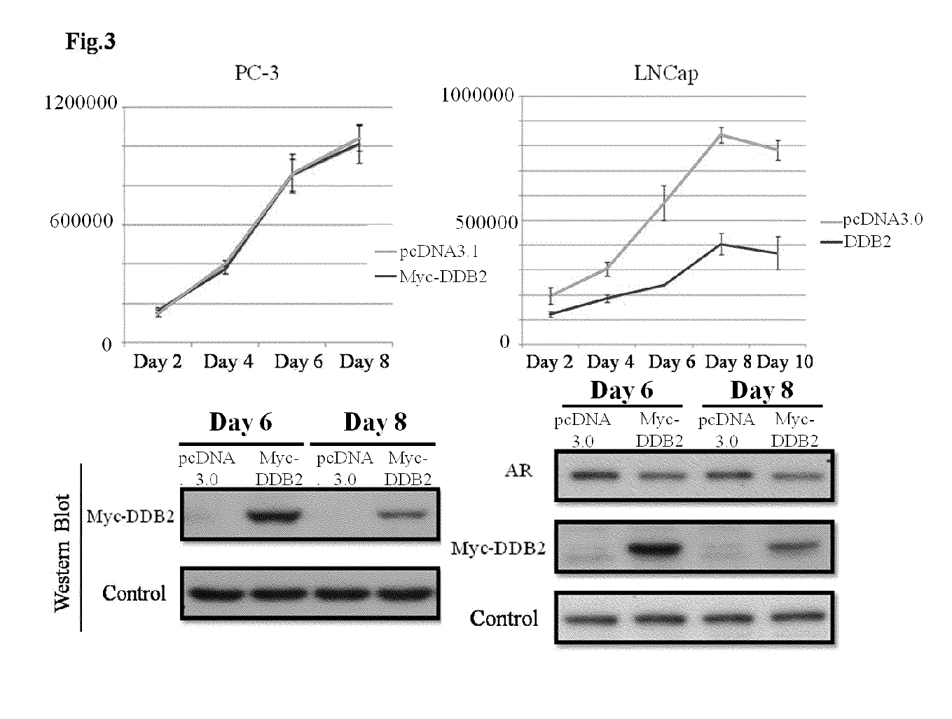

Two kinds of prostate cancer cells including LNCaP and PC-3 cells, which are androgen-dependent and androgen-independent respectively, were seeded into 24-well plates and transfected with Myc-DDB2, or pcDNA3.0 as a control by electroporation. After plating and maintaining the cells in the medium containing FBS, total cell numbers were counted every 2 days using a hemocytometer and trypan blue exclusion. The cell lysates at day 6 and day 8 were also collected and Western blotting was performed to detect the protein levels as indicated. The data shown in FIG. 3 demonstrate that DDB2 can degrade AR resulting in the decreased growth rate in AR-containing cells (LNCaP) but not in AR-null cells (PC-3), implying that DDB2 involves in AR protein degradation.

What is claimed is:

1. A method for treating a subject suffering from growth of androgen receptor (AR)-containing tumor cells, comprising administrating the subject an effective amount of damaged-DNA binding protein 2 (DDB2).

2. The method of claim 1, wherein the DDB2 interacts with androgen receptor (AR).

3. The method of claim 1, wherein the growth of AR-containing tumor cells are suppressed by degradation of AR.

4. The method of claim 3, wherein the degradation of AR is via ubiquitin mechanism.

5. The method of claim 4, wherein the ubiquitin mechanism involves participation of DDB1-CUL4A (cullin4A) complex.

6. The method of claim 1, wherein the subject is a mammal.

7. The method of claim 6, wherein the mammal is human.

8. The method of claim 1, wherein the tumor is prostate cancer.

9. The method of claim 1, wherein the administration is made by injections.

10. The method of claim 1, wherein the administration is made by oral route.

11. A cancer-diagnosing kit, comprising:
(a) DDB2, and
(b) DDB2-biomarker complex.

12. The cancer-diagnosing kit of claim 11, wherein the biomarker complex is selected from radioactive isotopes, fluorescent molecules, luminescent markers, enzymes or affinity molecules.

13. The cancer-diagnosing kit of claim 11, wherein the cancer-diagnosing kit further comprises a signal-detecting reagent.

14. The cancer-diagnosing kit of claim 13, wherein the signal-detecting reagent is selected from the group including antibodies, enzymes, affinity molecules and chemical coloring elements.

15. The cancer-diagnosing kit of claim 11, wherein the cancer is prostate cancer.
